(19) United States
(12) Patent Application Publication
(10) Pub. No.: US 2009/0054390 A1
(43) Pub. Date: Feb. 26, 2009

(54) Use of haptoglobin genotyping in diagnosis and treatment of intraplaque hemorrhage resulting from plaque rupture

(76) Inventors: Andrew Levy, Haifa (IL); Noah Berkowitz, New Rochelle, NY (US)

Correspondence Address: Pearl Cohen Zedek Latzer, LLP, 1500 Broadway, 12th Floor, New York, NY, 10036, US

(21) Appl. No.: 12/155,561

(22) Filed: Jun. 5, 2008

Related U.S. Application Data
(60) Provisional application No. 60/924,937, filed on Jun. 6, 2007.

(51) Int. Cl. A61K 31/395; C12Q 1/68; A61P 9/00

(52) U.S. Cl. 514/183; 435/6

(57) ABSTRACT

This invention relates to methods for providing prognosis of a subjects susceptibility to plaque rupture and compositions for treating plaque rupture and intraplaque hemorrhage. Specifically, the invention is directed to the use of haptoglobin genotyping in determining the susceptibility of a subject to develop intraplaque hemorrhage resulting from plaque rapture and treatment of the intraplaque hemorrhage using antioxidants.

### CROSS-REFERENCE TO RELATED APPLICATION

This application claims priority under 35 U.S.C. § 119(e) to provisional patent application Ser. No. 60/924,937 filed Jun. 6, 2007, which is hereby incorporated by reference in its entirety.

### FIELD OF THE INVENTION

This invention is directed to methods for providing prognosis of a subjects susceptibility to plaque rupture and compositions for treating plaque rupture Specifically, the invention is directed to the use of haptoglobin genotyping in determining the susceptibility of a subject to develop intraplaque hemorrhage resulting from plaque rapture and treatment of the intraplaque hemorrhage using antioxidants.

### BACKGROUND OF THE INVENTION

Atherosclerotic coronary artery disease is the leading cause of death in industrialized countries. Typically, patients who have died of coronary disease may exhibit as many as several dozen atherosclerotic plaques in the arterial tree. Plaque, a thickening in the arterial vessel wall, results from the accumulation of cholesterol, proliferation of smooth muscle cells, secretion of a collagenous extracellular matrix by the cells, and accumulation of inflammatory cells and, eventually, hemorrhage, thrombosis and calcification. Pathological features of high risk plaques include: a lipid core containing substantial free and esterified cholesterol, and other necrotic debris; infiltrated macrophages (and less frequently lymphocytes, monocytes and mast cells); less abundant smooth muscle cells; and, consequentially, low content of collagen, other matrix proteins and intraplaque hemorrhage.

Extracorpuscular hemoglobin (Hb) released from red blood cells after intra-plaque hemorrhage represents a potent stimulus for inflammation within the plaque. It is becoming apparent that the frequency of microvascular hemorrhages has been severely underestimated and may occur in up to 40% of all advanced atherosclerotic plaques.

An important defense mechanism to counteract the effects of intra-plaque hemorrhage is mediated by haptoglobin (Hp), an abundant serum protein whose primary function is to bind to extracorpuscular Hb, thereby attenuating its oxidative and inflammatory potential. Hp also promotes the clearance of extracorpuscular Hb via the CD163 scavenger receptor present on macrophages. This scavenging pathway is the only mechanism that exists for removing free Hb released at extravascular sites, i.e., at sites of hemorrhage within the atherosclerotic plaque.

In humans there exist 2 classes of alleles for Hp, designated 1 and 2. The Hp to polymorphism is a common polymorphism. In the western world, 16% of the population is Hp 1-1 (homozygous for the Hp 1 allele), 36% is Hp 2-2 (homozygous for the Hp 2 allele), and 48% is Hp 2-1 (heterozygote). The Hp 2 allele is found only in humans. All other mammals, including higher primates have only the Hp 1 allele and therefore have the Hp 1-1 genotype. The Hp 2 allele appears to have been generated by an intragenic duplication event is of exons 3 and 4 of the Hp 1 allele ˜100 000 years ago early in human evolution.

The lipid core, which is mainly a large pool of cholesterol, characterizing most ruptured plaque, results from insudation and from the release of the contents of foam cells following degradation of the cell wall. The low content of collagen and matrix proteins associated with at-risk plaque contributes to an important feature of the unstable plaque—the thin plaque cap. The release of matrix-digesting enzymes by the inflammatory cells is thought to contribute to plaque rupture. Small blood clots, particularly microthrombi, are also frequently found on non-ruptured but inflamed ulcerated plaque surfaces.

Due to the mortality and morbidity associated with plaque rupture and the resulting hemorrhagic events there continues to exist a need for effective prognosis of risk factors and treatments that are based on the prognosis.

### SUMMARY OF THE INVENTION

In one embodiment, the invention provides a method of determining susceptibility of a subject to a plaque rupture comprising the step of obtaining a biological sample from the subject; and determining the subject's haptoglobin allelic genotype, whereby a subject expressing the Hp-2-2 genotype is susceptible to plaque rupture.

In another embodiment, the invention provides a method of treating a plaque rupture in a subject, comprising the step of contacting the subject with an effective amount of a composition comprising an antioxidant or its isomer, metabolite, and/or salt therefore, thereby treating plaque rupture.

In one embodiment, the invention provides a method of inhibiting or suppressing a plaque rupture in a subject comprising the step of contacting the subject with an effective amount of a composition comprising an antioxidant or its isomer, metabolite, and/or salt therefore,thereby inhibiting or suppressing plaque rupture.

In another embodiment, the invention provides a method of reducing symptoms associated with a plaque rupture in a subject comprising the step of contacting the subject with an effective amount of a composition comprising an antioxidant or its isomer, metabolite, and/or salt therefore, thereby reducing symptoms associated with plaque rupture.

In one embodiment, the invention provides a method of treating an intraplaque hemorrhage in a subject, comprising the step of contacting the subject with an effective amount of a composition comprising an antioxidant or its isomer, metabolite, and/or salt therefore, thereby treating intraplaque hemorrhage.

In another embodiment, the invention provides a method of inhibiting or suppressing an intraplaque hemorrhage in a subject comprising the step of contacting the subject with an effective amount of a composition comprising an antioxidant or its isomer, metabolite, and/or salt therefore, thereby inhibiting or suppressing intraplaque hemorrhage.

In one embodiment, the invention provides a method of reducing symptoms associated with an intraplaque hemorrhage in a subject comprising the step of contacting the subject with an effective amount of a composition comprising an antioxidant or its isomer, metabolite, and/or salt therefore, thereby reducing symptoms associated with intraplaque hemorrhage.

In one embodiment, the invention provides a method of determining susceptibility of a subject to a atherosclerosis comprising the step of obtaining a biological sample from the subject; and determining the subject's haptoglobin allelic genotype, whereby a subject expressing the Hp-2-2 genotype is susceptible to atherosclerosis.

In another embodiment, the invention provides a method of treating atherosclerosis in a subject, comprising the step of contacting the subject with an effective amount of a composition comprising an antioxidant or its isomer, metabolite, and/or salt therefore, thereby treating plaque rupture.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows the construction of a murine Hp 2 allele. A, Genomic organization of the Hp locus. The human Hp 1 and Hp 2 alleles are located at chromosomal coordinates 16q22. The murine wild type Hp is a Hp 1 allele and is found on chromosome 8. A murine Hp 2 allele was created as described in this manuscript and inserted by homologous recombination at the wild type Hp locus replacing the murine Hp 1 allele. In the human Hp 2 allele, exons 5 and 6 represent a duplication of exons 3 and 4. The mouse Hp 1 allele has the identical intron-exon boundaries as the human Hp 1 allele and is 90% homologous at the amino acid level. The murine Hp 2 allele, constructed as described in the text, is similar to the human Hp 2 allele in that it has a direct repeat of exons 3 and 4. The exonic organization of the human and murine Hp 2 alleles are identical after RNA splicing has occurred. B, Fine map of the murine Hp locus before and after gene targeting. Top, Genomic organization of the murine Hp 1 allele. B, Bam H1; Bg, BglII; E, EcoR1; P, PvuII. Middle, Genomic organization of the murine Hp 2 allele after successful gene targeting by homologous recombination. A targeting vector was constructed using the pTKLNCL GB 135 vector as a backbone. TKLNCL contains lox P sites (large arrow) bracketing the gene for cytosine deaminase (CD) and the neomycin (Neo) resistance gene. A 5.8-kb E-P fragment of the murine Hp 1 allele was cloned in the KpnI-XhoI site of TKLNCL 5′ to the neo cassette (5′ homology region) and a 3.4 kb BglII fragment of the murine Hp 1 allele was cloned in the Bam H1 site of TKLNCL 3′ to the neo cassette (3′ homology region). Exon 3 of the murine Hp 1 was reconstructed to be exon 343 as described in Methods. The vector was linearized with NotI before transfection. Identification of G418 resistant ES clones that integrated the targeting vector at the Hp locus by homologous recombination was achieved by Southern blot analysis of Bam H1 digested DNA from these clones using a 300-bp BamH1-BglII fragment (in blue) as probe. This probe hybridizes with a 5.8 kb Bam H1 fragment in wild type DNA (Hp 1) and with a 11 kb Bam H1 fragment in successfully targeted clones (Hp 2) (shown in FIG. 1 of online supplement). Bottom, Genomic organization of the murine Hp 2 allele after removal of the Neo and CD cassettes with cre recombinase;

FIG. 2 shows that the size and shape of murine Hp 2 polymers are similar to human Hp 2 polymers. A, Schematic illustration of the shape of Hp polymers in humans with the Hp 1-1, Hp 2-1 or Hp 2-2 genotypes. The Hp monomer forms multimers whose stoichiometry is Hp genotypedependent. Multimerization is mediated by cysteine residues in exon 3 so that the Hp 1 allele protein product can combine with only one other monomer while the Hp 2 allele protein product combines with 2 other monomers. The structures shown have been verified by electron microscopy. B, Demonstration that the polymer distribution in murine Hp 1-1, 2-1, and 2-2 mice is similar to that in humans with Hp 1-1, 2-1, and 2-2. Shown is a polyacrylamide gel of serum samples from humans or mice with the indicated Hp genotypes. Samples were enriched with Hb and then electrophoresed on a nondenaturing polyacrylamide gel. Hp-Hb complexes were identified in the gel using a peroxidase sensitive reagent. A signature banding pattern is present for each Hp genotype. Note that higher molecular Hp-Hb complexes are absent in Hp 1-1 mice and that the distribution of the high-molecular-weight complexes in murine Hp 2-1 and Hp 2-2 mice is quite similar to that in humans with Hp 2-1 and Hp 2-2. Both the human Hp 1-1-Hb complex and the murine Hp 1-1-Hb complex are a single species (demarcated with an asterisk*) located just above the free Hb band;

FIG. 3 shows increased iron in plaques from Hp 2-2 mice. Intraplaque iron is stained black (representative examples noted with arrows) with Perl's stain. The amount of iron staining in plaques from Hp 2-2 ApoE^{-/-} mice was significantly greater than in Hp 1-1 ApoE^{-/-} mice when scored as the percentage of the total plaque area (2.18±0.26% vs 0.94±0.25%, n=10, P=0.008);

FIG. 4 shows increased lipid peroxidation in plaques of Hp 2-2 mice. A, Increased 4-HNE in plaques of Hp 2-2 mice. 4-HNE protein adducts (staining brown) in the plaque were assessed by immunohistochemistry as described in Methods. B, Increased ceroid (autofluorescence) in plaques of Hp 2-2 mice. The autofluorescent ceroid pigment (arrow) in the plaque was scored as the percentage of ceroid (autofluorescence) of the total plaque area. There was significantly more ceroid in Hp 2-2 plaques as compared with Hp 1-1 plaques (10.3±3.9% vs 2.6±0.5% of total plaque area, n=8, P=0.05); and

FIG. 5 shows increased macrophage accumulation in the plaques of Hp 2-2 mice. Macrophages were identified immunohistochemically as described in methods. Shown in (A) and (B) are representative plaques of similar size but with dramatically greater macrophage accumulation in Hp 2-2 ApoE^{-/-} (A) as compared with Hp 1-1 ApoE^{-/-} (B) mice. C, Histogram of the mean_SEM of the number of macrophages in the intima and adventitia from all plaques (n=18 for Hp 1-1 and n=15 for Hp 2-2). There were significantly more macrophages in the intima (P=0.03) and adventitia (P_{—}0.03) of plaques from Hp 2-2 as compared with Hp 1-1 mice. D, Plot of the number of intimal macrophages versus the lipid core area (μm²) in plaques from Hp 1-1 ApoE^{-/-} (n=18) and Hp 2-2 ApoE^{-/-} (n=15) mice. There was a statistically significant correlation between the number of macrophages and the lipid core area in plaques from Hp 2-2 mice (correlation coefficient=0.57, P=0.01) but not in Hp 1-1 mice (correlation coefficient=0.08, P=0.38).

### DETAILED DESCRIPTION OF THE INVENTION

In one embodiment the Hp 2-2 genotype is associated with an increased risk of atherosclerotic cardiovascular disease and its sequelae such as acute myocardial infarction. In another embodiment, the differences in the antioxidant and immunomodulatory properties of the Hp 1-1 and Hp 2-2 proteins make Hp a susceptibility gene for cardiovascular disease (CVD). As an antioxidant the Hp 1-1 protein is superior to the Hp 2-2 protein in blocking the oxidative action of Hb. As an immunomodulator, the Hp 1-1-Hb complex stimulates in another embodiment the macrophage to secrete anti-inflammatory cytokines to a markedly greater degree than the Hp 2-2-Hb complex.

In one embodiment, the Hp genotype specifies the nature and intensity of the macrophage response to intraplaque hemorrhage and thereby serves as a determinant of susceptibility to plaque rupture.

Accordingly and in one embodiment, provided herein is a method of determining susceptibility of a subject to a plaque rupture comprising the step of obtaining a biological sample from the subject; and determining the subject's haptoglobin allelic genotype, whereby a subject expressing the Hp-2-2 genotype is susceptible to, or at risk for, plaque rupture.

Accordingly and in one embodiment, provided herein is a method of determining susceptibility of a subject to a atherosclerosis comprising the step of obtaining a biological sample from the subject; and determining the subject's haptoglobin allelic genotype, whereby a subject expressing the Hp-2-2 genotype is susceptible to, or at risk for, atherosclerosis.

In one embodiment, the term “plaque rupture” refers to an area of fibrous cap disruption whereby the overlying thrombus is in continuity with the underlying necrotic core. In another embodiment, ruptured lesions have a large necrotic core and a disrupted fibrous cap infiltrated by macrophages and lymphocytes. In another embodiment, the term “plaque rupture” refers to superficial erosion. The term “superficial erosion” refers in another embodiment to a thrombus confined to the most luminal portion of a fibrous cap in the absence of fissure or rupture after serial sectioning.

The term “fibrous cap” refers in one embodiment, to a distinct layer of connective tissue completely covering the lipid core. In another embodiment, the fibrous cap consists purely of smooth muscle cells in a collagenous proteoglycan matrix, with varying degrees of infiltration by macrophages and lymphocytes. Thus, in one embodiment, a fibrous cap atheroma has a thick or thin cap overlying a lipid-rich core. As plaque lesions progress, the core of necrotic debris surrounded by macrophages becomes increasingly consolidated in another embodiment, into one or more masses comprising large amounts of extracellular lipid, cholesterol crystals, and necrotic debris.

In one embodiment, lesions with thin, fibrous caps are likely to rupture. In another embodiment, lesions with thin fibrous caps in subjects exhibiting the Hp-2-2 allele, are the most likely to rupture. Accordingly and in one embodiment, subjects exhibiting thin fibrous cap and Hp 2-2 allele are at a high risk of plaque rupture.

Plaque rupture is triggered in one embodiment, by mechanical events, but plaque vulnerability is due to weakening of the fibrous cap in another embodiment, or intraplaque hemorrhage, softening of plaque components, often as a result of infection and macrophage, T-cell infiltration or their combination in other embodiments. In one embodiment, lipid-rich, soft plaques are more prone to rupture than collagen-rich, hard plaques to rupture. Several morphological and physiological features are associated with vulnerable and stable plaque. Morphological characteristics suggest structural weakness or damage (thin or ruptured fibrous cap, calcification, negative remodeling, neovascularization, large lipid deposits, etc.), while physiological features suggest chemical composition, active infection, inflammatory responses, and metabolism. In one embodiment, haptoglobin genotype is associated with plaque vulnerability and its determination in another embodiment, using the methods provided herein is used in its therapy and treatment.

In one embodiment, plaque rupture results from the critical effects of inflammation whereby cytokines drive the expression of proteases and obstruct the actions of proteolytic inhibitors. In another embodiment, plaque rupture is caused by specific antigens, which elicit a T-cell response whereby disease progression is stimulated by autoimmune responses to oxidized lipoproteins.

In one embodiment, the term “at-risk”, “vulnerable,” “dangerous” or “unstable” plaque are interchangeable and refer to an atherosclerotic plaque which, in a living vessel, is likely to develop a fissure, rupture or develop a thrombus leading to a life-threatening event.

In one embodiment, in instances of myocardial infarction, or cardiac arrest, and perhaps stroke in other embodiments, it is only one of an artery's many lesions (plaques) which has actually ruptured, fissured, or ulcerated. In another embodiment, the rupture, fissure, or ulcer causes a large thrombus (blood clot) to form on the inside of the artery. Such a large blood clot occludes the flow of blood through the artery in one embodiment, causing injury to the heart or brain. In one embodiment, unstable coronary atherosclerotic plaques occur in arteries with 50% or less luminal diameter narrowing.

In another embodiment, red blood cell (RBC) membranes are rich in phospholipids and free cholesterol, and their accumulation within plaques plays a key role in promoting lesion instability through necrotic core expansion in one embodiment, inflammatory cell infiltration or both in another embodiment. The source of RBCs within coronary lesions is provided in one embodiment, by inherently leaky immature blood vessels that surround and invade the plaque. In another embodiment, extracorpuscular hemoglobin (Hb) released from red blood cells after intra-plaque hemorrhage is a potent stimulus for inflammation within the plaque. Haptoglobin (Hp), binds in one embodiment to extracorpuscular Hb, thereby attenuating its oxidative and inflammatory potential. In another embodiment, Hp promotes the clearance of extracorpuscular Hb via the CD163 scavenger receptor present on macrophages. Accordingly, any differential capability of Hp alleles to perform the functions described hereinabove, will have an effect on the vulnerability of plaque to tupture and the severity of the resulting hemorrhage.

In one embodiment, plaque rupture is the principal cause of luminal thrombosis in acute coronary syndromes, occurring in about 75% of patients dying of an acute myocardial infarction (MI). In another embodiment, plaques vulnerable to rupture are characterized by the same histopathologic signatures, as stable plaque except that they still have an intact fibrous cap.

In one embodiment macrophage infiltration is the first step toward the eventual formation of an atherosclerotic plaque. Low-density lipoprotein (LDL) uptake by macrophages is facilitated in another embodiment by a 2-step oxidation process, beginning with mild oxidation of lipid, followed by apo-lipoprotein B oxidation, a modification required for scavenger receptor recognition, which is unaffected by the cholesterol content of the cell. The threshold level of free cholesterol in macrophages is regulated in one embodiment by a re-esterification process involving acyl coenzyme A:acylcholesterol transferase, (ACAT1). Formation of necrotic core is attributed in one embodiment to the death of macrophages. As plaques progress from fatty streaks to those with necrotic cores (gruel plaques), the free cholesterol content of the lesion increases, whereas cholesterol esters decrease. In one embodiment, the increase in free cholesterol is associated with lesion instability. In one embodiment, Hp genotype affects LDL uptake by macrophages, thereby affecting the formation of atherosclerotic plaque.

Haptoglobin is inherited by two co-dominant autosomal alleles situated on chromosome 16 in humans, these are Hp1 and Hp2. There are three phenotypes Hp1-1, Hp2-1 and Hp2-2. The haptoglobin molecule is a tetramer comprising of four polypeptide chains, two alpha and two beta chains, of which alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp1-1 individuals have greater hemoglobin binding capacity when compared to those individuals with Hp2-1 and Hp2-2. The gene differentiation to Hp-2 from Hp-1 resulted in a dramatic change in the biophysical and biochemical properties of the haptoglobin protein encoded by each of the 2 alleles.

In one embodiment, Hp-Hb complex in plaque is derived from two possible routes, both of which are increased in diabetes mellitus (DM): in one embodiment, extravasation from plasma and in another embodiment from intraplaque hemorrhage. In the plaque embodiment, Hp 2-Hb complexes, particularly in the setting of DM are scavenged at a much slower rate than Hp 1-Hb complexes resulting in a much higher concentration of Hp-Hb complex in Hp 2 DM plaques. In one embodiment, Hp-2-Hb complex in the plaque promote a pro-inflammatory macrophage phenotype via oxidative mechanisms leading in another embodiment, to plaque destabilization. In another embodiment, the Hp-1-Hb complex promotes an anti-inflammatory macrophage phenotype via interaction with CD163 leading to plaque stabilization. Accordingly and in one embodiment, upon determination of the subject Hp genotype, therapies are tailored for the patient's allelic expression. In one embodiment, antioxidants targeted to Hp 2 DM individuals provide a considerable cardiovascular benefit.

Lipoproteins have in one embodiment, the function of transporting lipids throughout the body. Low density lipoproteins are responsible in another embodiment, for the transport of cholesterol with the protein moiety involved: apolipoprotein (Apo) B. Very low density lipoproteins are responsible in one embodiment, for the transport of triglycerides with the protein moiety involved: Apo E. In another embodiment, HDLs are responsible for reverse cholesterol transport and in one embodiment, play an important role in being a naturally occurring potent anti-inflammatory and antioxidant agent with the protein moiety involved: Apo A. It is the protein moiety of the lipoproteins that is modified in one embodiment, by the processes of oxidation, glycation, and glycoxidation with a resultant increase in redox stress and the production of ROS. In one embodiment, the modification of the protein moiety is responsible for their retention within the intima, inducing in one embodiment, atherogenesis and thus atheroscleropathy. Accordingly and in one embodiment, Hp genotype is predictive of the extent of glycoxidation capable of modifying Apo A, thereby leading to increased redox stress, wherein the extent of glycoxidation or in one embodiment, oxidation, decreases from Hp-2-2, to Hp-2-1, to Hp-1-1, and is diagnosed according to the methods provided herein.

In one embodiment, antioxidant therapy may be beneficial in specific subgroups with increased oxidative stress. Oxidative stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues such as those resulting from intraplaque hemorrhage, must be able to protect themselves against the toxic effects of ROS. In one embodiment ROS-detoxifying enzymes have an important role in epithelial wound repair. In another embodiment, the glutathione peroxidase mimetics provided in the compositions and compounds provided herein, replace the ROS detoxifying enzymes described herein.

In one embodiment, overproduction of reactive oxygen species (ROS) including hydrogen peroxide (H₂O₂), superoxide anion (O.₂⁻); nitric oxide (NO.) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPX) and catalase are, involved in the disposal of O.₂⁻ and H₂O₂. First, SOD catalyses the dismutation of O.₂⁻ to H₂O₂ and molecular oxygen (O₂), resulting in selective O.₂⁻ scavenging. Then, GPX and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When free radicals are generated in excess or when FRSEs are defective, H₂O₂ is reduced into hydroxyl radical (OH.), which is one of the highly reactive ROS responsible in one embodiment for initiation of lipid peroxidation of cellular membranes. In another embodiment, organic peroxide-induced lipid peroxidation is implicated as one of the essential mechanisms of toxicity in the death of hippocampal neurons. In one embodiment, an indicator of the oxidative stress in the cell is the level of lipid peroxidation and its final product is MDA. In another embodiment the level of lipid peroxidation increases in inflammatory diseases, such as meningitis in one embodiment. In one embodiment, the compounds provided herein and in another embodiment, are represented by the compounds of formula I-X, are effective antioxidants, capable of reducing lipid peroxidation, or in another embodiment, are effective as anti-inflammatory agents.

According to various typical embodiments of the method of the present invention, determining the haptoglobin phenotype of a subject is effected by any one of a variety of methods including, but not limited to, a signal amplification method, a direct detection method and detection of at least one sequence change. These methods determine a phenotype indirectly, by determining a genotype. As will be explained hereinbelow, determination of a haptoglobin phenotype may also be accomplished directly by analysis of haptoglobin gene products.

Haptoglobin is inherited by two co-dominant autosomal alleles situated on chromosome 16 in humans, these are Hp1 and Hp2. There are three phenotypes Hpl-1, Hp2-1 and Hp2-2. Haptoglobin molecule is a tetramer comprising of four polypeptide chains, two alpha and two beta chains, of which alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp1-1 individuals have greater hemoglobin binding capacity when compared to those individuals with Hp2-1 and Hp2-2. The gene differentiation to Hp-2 from Hp-1 resulted in a dramatic change in the biophysical and biochemical properties of the haptoglobin protein encoded by each of the 2 alleles. The gene differentiation to Hp-2 from Hp-1 resulted in a dramatic change in the biophysical and biochemical properties of the haptoglobin protein encoded by each of the 2 alleles. The haptoglobin phenotype of any individual, 1-1, 2-1 or 2-2, is readily determined in one embodiment, from 10 μl of plasma by gel electrophoresis.

The signal amplification method according to various preferred embodiments of the present invention may amplify, for example, a DNA molecule or an RNA molecule. Signal amplification methods which might be used as part of the present invention include, but are not limited to PCR, LCR (LAR), Self-Sustained Synthetic Reaction (3SR/NASBA) or a Q-Beta (Qβ) Replicase reaction.

In another embodiment, the methods and compositions provided herein, for determining susceptibility of a subject to a plaque rupture comprising the step of obtaining a biological sample from the subject; and determining the subject's haptoglobin allelic genotype, whereby a subject expressing the Hp-2-2 genotype will bvulnerable to, or at risk for plaque rupture, is effected by a signal amplification method, whereby said signal amplification method is PCR, LCR (LAR), Self-Sustained Synthetic Reaction (3SR/NASBA), Q-Beta (Qβ) Replicase reaction, or a combination thereof.

In another embodiment, the methods and compositions provided herein, for determining susceptibility of a subject to a atherosclerosis and benefit from therapy, comprising the step of obtaining a biological sample from the subject; and determining the subject's haptoglobin allelic genotype, whereby a subject expressing the Hp-2-2 genotype will be vulnerable to, or at risk for atherosclerosis, or benefit from therapy therefor.

In another embodiment, the signal amplification methods provided herein, which in another embodiment, can be carried out using the systems provided herein, may amplify a DNA molecule or an RNA molecule. In another embodiment, signal amplification methods used as part of the present invention include, but are not limited to PCR, LCR (LAR), Self-Sustained Synthetic Reaction (3SR/NASBA) or a Q-Beta (Qβ) Replicase reaction.

Polymerase Chain Reaction (PCR): The polymerase chain reaction (PCR), refers in one embodiment to a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, in one embodiment, they are said to be “PCR-amplified.”

Ligase Chain Reaction (LCR or LAR): The ligase chain reaction [LCR; referred to, in another embodiment as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed in one embodiment and DNA ligase is added to the mixture. Provided that there is complete complementarity at the junction, ligase will covalently link each set of hybridized molecules. In another embodiment of LCR, two probes are ligated together only when they base-pair with sequences in the target sample, without gaps or mismatches. Repeated cycles of denaturation, and ligation amplify a short segment of DNA. LCR has is used in combination with PCR in one embodiment, to achieve enhanced detection of single-base changes. In another embodiment, because the four oligonucleotides used in this assay can pair to form two short ligatable fragments, there is the potential for the generation of target-independent background signal. The use of LCR for mutant screening is limited in another embodiment, to the examination of specific nucleic acid positions.

Self-Sustained Synthetic Reaction (3SR1NASBA): The self-sustained sequence replication reaction (3SR) refers in one embodiment, to a transcription-based in vitro amplification system that can exponentially amplify RNA sequences at a uniform temperature. The amplified RNA is utilized in certain embodiments, for mutation detection. In an embodiment of this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ.) Replicase: In one embodiment of the method, a probe which recognizes the sequence of interest is attached to the replicatable RNA template for Qβ. replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

In another embodiment, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

In another embodiment, the methods provided herein for determining susceptibility of a subject to a plaque rupture comprising the step of obtaining a biological sample from the subject; and determining the subject's haptoglobin allelic genotype, whereby a subject expressing the Hp-2-2 genotype will bvulnerable to, or at risk for plaque rupture, is effected by a direct detection method such as a cycling probe reaction (CPR), or a branched DNA analysis, or a combination thereof in other embodiments.

The direct detection method according to one embodiment is a cycling probe reaction (CPR) or a branched DNA analysis. When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

In one embodiment, the methods provided herein for determining susceptibility of a is subject to a plaque rupture comprising the step of obtaining a biological sample from the subject; and determining the subject's haptoglobin allelic genotype, whereby a subject expressing the Hp-2-2 genotype will bvulnerable to, or at risk for plaque rupture, is effected by at least one sequence change, which employs in one embodiment a restriction fragment length polymorphism (RFLP analysis), or an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF) or their combination in other embodiments.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perhnan and Butow, Science 246:1106, 1989), but again, these are few in number.

Allele specific oligonucleotide (ASO): allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Determination of a haptoglobin phenotype may, as is further exemplified in the Examples section that hereinbelow, may be accomplished directly in one embodiment, by analyzing the protein gene products of the haptoglobin gene, or portions thereof. Such a direct analysis is often accomplished using an immunological detection method. In one embodiment, the methods and systems provided herein for providing a prognosis for development of a diabetic subject to benefit from supplementation of vitamin-E, comprising the steps of: obtaining a biological sample from a subject; determining the Haptoglobin (Hp) genotype in the biological sample by an immunological detection method, such as is a radio-immunoassay (RIA) in one embodiment, or an enzyme linked immunosorbent assay (ELISA), a western blot, an immunohistochemical analysis, or fluorescence activated cell sorting (FACS), or a combination thereof in other embodiments.

Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two haptoglobin alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, haptoglobin in this case and in the methods detailed hereinbelow, with a specific antibody and radiolabelled antibody binding protein (e.g., protein A labeled with I.sup.125) immobilized on a precipitable carrier such as agarose beads. The number of counts in the precipitated pellet is proportional to the amount of substrate. In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the haptoglobin phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

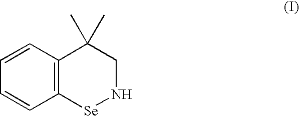

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of 7 electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet. In vitro and in vivo studies with the compound of formula 1, a show in one embodiment, that glutahion peroxidase or its isomer, metabolite, and/or salt therefore is capable of protecting cells against reactive oxygen species.

In one embodiment, the antioxidants used in the methods described herein are small-molecule antioxidants and antioxidant enzymes. Suitable small-molecule antioxidants include, in another embodiment, hydralazine compounds, glutathione, vitamin C, vitamin E, cysteine, N-acetyl-cysteine, β-carotene, ubiquinone, ubiquinol-10, tocopherols, coenzyme Q, and the like. Suitable antioxidant enzymes are in one embodiment superoxide dismutase (SOD), or catalase, glutathione peroxidase, or a combination thereof in other embodiments. Suitable antioxidants are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996.

In one embodiment, the therapeutic value of the compositions provided herein, is effected by administration of recognized antioxidant free radical trapping compounds such as α-tocopherol, edaravone or other co-agents previously recognized as adjuncts which facilitate in vivo capability to inhibit lipid peroxidation in one embodiment.

In one embodiment agents which function to supplement the chain-breaking antioxidant property of vitamin E are ubiquinol in one embodiment, or seleno-amino acids and sulfhydryl compounds (e.g., glutathione, sulfhydryl proteins, cysteine and methionine) in other embodiments. Other substances in this general group include in other embodiments: butylated hydroxytoluene (BHT), butylated hydroxyanisole (BHA), propyl gallate (PG), dodecylgallate, tert-butylhydroquinone (TBHQ), dihydrolipoic acid, prostaglandin B₁ oligomers (also known as polymeric 15-keto prostaglandin B or PGBₓ), 2-aminomethyl-4-tert-butyl-6-iodophenol, 2-aminomethyl-4-tert-butyl-6-propionylphenol, 2,6-di-tert-butyl-4-[2′-thenoyl]phenol, N,N′-diphenyl-p-phenylenediamine, ethoxyquin, probucol and its derivative such as AGI-1067, 5-[[3,5-bis(1,1-dimethylethyl)-4-hydroxyphen-yl]methylene]-3-(dimethylamino)-4-thiazolidinone (LY221068), 5-[[3,5-bis(1,1-dimethylethyl)-4-hydroxyphenyl]meth-ylene]-3-(methylamino)-4-thiazolidinone (LY269415), D-myoinositol-1.2.6-trisphosphate, nordihydroguaiaretic acid, deferoxamine mesylate, tirilazad mesylate (U-74006F), derivative of tirilazad in which the steroid portion of the chemical structure has been replaced with the tetramethyl chroman portion of d-α-tocopherol (U78517F), trimetazidine, N,N′-dimethylthiourea, 2-(2-hydroxy-4-methylphenyl)amino-thiazole-hydrochloride, or 2-L-oxothiazolidine. In one embodiment, any of the antioxidants described herein may be used in the methods described herein.

In another embodiment, Thioctic acid, also known as α-lipoic acid, is used as an antioxidant in the compositions and methods provided herein, including its sodium salt and ethylenediamine derivatives. In one embodiment, antioxidants and free radical trapping substances used in the compositions and methods provided herein, are plant (e.g., vegetable) active ingredients. This category, includes in one embodiment parthenolide, or lycopene, genistein, quercetin, morin, curcumin, apigenin, sesamol, chlorogenic acid, fisetin, ellagic acid, quillaia saponin, capsaicin, ginsenoside, silymarin, kaempferol, ginkgetin, bilobetin, isoginkgetin, isorhamnetin, herbimycin, rutin, bromelain, levendustin A, orerbstatin in other embodiments.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of GPx or a mmetic thereof, its pharmaceutically acceptable salt, its functional derivative, its synthetic analog or a combination thereof, is used in the methods and compositions of the invention.

Accordingly and in one embodiment, provided herein is a method of treating plaque rupture in a subject, or in another embodiment, inhibiting or suppressing plaque rupture in a subject, or in another embodiment, reducing symptoms associated with plaque rupture in a subject; comprising the step of contacting the subject with an effective amount of a composition comprising an antioxidant or its isomer, metabolite, and/or salt therefore, and cholesteryl ester transfer protein inhibitor thereby plaque rupture, or in another embodiment, intraplaque hemorrhage.

In another embodiment, provided herein is a method of treating atherosclerosis in a subject, or in another embodiment, inhibiting or suppressing the development of atherosclerosis in a subject, or in another embodiment, reducing symptoms associated with atherosclerosis in a subject; comprising the step of contacting the subject with an effective amount of a composition comprising an antioxidant or its isomer, metabolite, and/or salt therefore. In another embodiment, provided herein is a method of treating atherosclerosis in a subject with the Hp 2-2 genotype, or in another embodiment, inhibiting or suppressing the development of atherosclerosis in a subject with the Hp 2-2 genotype, or in another embodiment, reducing symptoms associated with atherosclerosis in a subject with the Hp 2-2; comprising the step of contacting the subject with an effective amount of a composition comprising an antioxidant or its isomer, metabolite, and/or salt therefore. In another embodiment, the subject is first tested for the presence of the Hp 2-2 genotype and subsequently administered the aforementioned composition. In another embodiment, the antioxidant is any of the compounds of formula I-X described herein.

In one embodiment, the antioxidant used in the methods of treating a plaque rupture in a subject comprising the step of contacting the subject with an effective amount of a composition comprising an antioxidant or its isomer, metabolite, and/or salt therefore; is glutathione peroxidase mimetic represented by formula I:

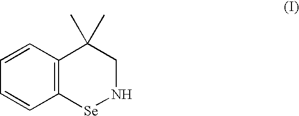

In one embodiment, the compound of formula (II), refers to benzisoselen-azoline or -azine derivatives of glutathione peroxidase mimetic and is represented by the following general formula:

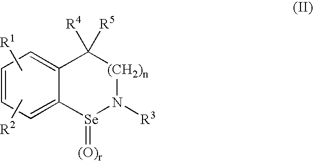

where: R¹, R²=hydrogen; lower alkyl; OR⁶; —(CH₂)ₘ NR⁶R⁷; —(CH₂)_{q}NH₂; —(CH₂)ₘ NHSO₂ (CH₂)₂ NH₂; —NO₂; —CN; —SO₃ H; —N⁺ (R⁵)₂O⁻; F; Cl; Br; I; —(CH₂)ₘR⁸; —(CH₂)ₘ COR⁸; —S(O)NR⁶ R⁷; —SO₂ NR⁶ R⁷; —CO(CH₂)ₚ COR⁸; R⁹; R³=hydrogen; lower alkyl; aralkyl; substituted aralkyl; —(CH₂)ₘ COR⁸; —(CH₂)_{q}R⁸; —CO(CH₂)ₚ COR⁸; —(CH₂)ₘ SO₂ R⁸; —(CH₂)ₘ S(O)R⁸; R⁴=lower alkyl; aralkyl; substituted aralkyl; —(CH₂)ₚ COR⁸; —(CH₂)ₚR⁸; F; R⁵=lower alkyl;aralkyl; substituted aralkyl; R⁶=lower alkyl;aralkyl; substituted aralkyl; —(CH₂)ₘCOR⁸; —(CH₂)_{q}R⁸; R⁷=lower alkyl;aralkyl; substituted aralkyl; —(CH₂)ₘCOR⁸; R⁸=lower alkyl;aralkyl; substituted aralkyl; aryl; substituted aryl; heteroaryl; substituted heteroaryl; hydroxy;lower alkoxy; R⁹; R⁹=

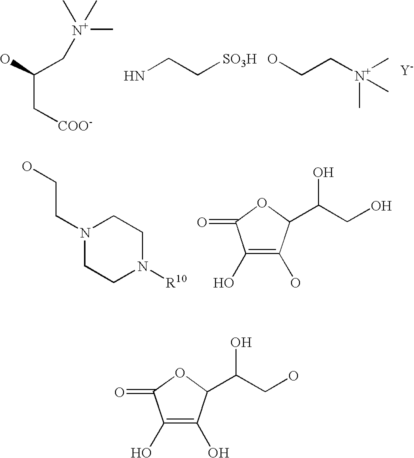

R¹⁰=hydrogen; lower alkyl;aralkyl or substituted aralkyl; aryl or substituted aryl;. Y⁻ represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl). or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —ORₓ; —SRₓ; —S(O)Rₓ; —S(O)2Rₓ; —NRₓ(CO)Rₓ; —N(Rₓ))CO₂Rₓ; —N(Rₓ)S(O)₂Rₓ; —N(Rₓ)C(O)N(Rₓ)₂; —S(O)₂N(Rₓ)₂; wherein each occurrence of Rₓ, independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety.

In one embodiment, the glutathione peroxidase or its isomer, metabolite, and/or salt therefore, used in the methods and compositions provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating plaque rupture, or in another embodiment, intraplaque hemorrhage, comprising organoselenium compounds, thereby increasing endogenous anti-oxidant ability of the cells, or in another embodiment, scavenging free radicals causing apoptosis of macrophages and their associated pathologies.

In another embodiment, the glutathione peroxidase or its isomer, metabolite, and/or salt therefore used in the compositions and methods provided herein, is represented by the compound of formula III:

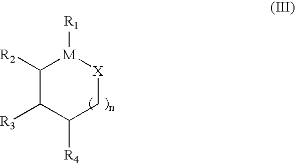

wherein,

the compound of formula 1 is a ring; and
X is O or NH
M is Se or Te
n is 0-2
R₁ is oxygen; and
forms an oxo complex with M; or
R₁ is oxygen or NH; and
   forms together with the metal, a 4-7 member ring, which optionally is substituted by an oxo group; or
   forms together with the metal, a first 4-7 member ring, which is optionally substituted by an oxo group, wherein said first ring is fused with a second 4-7 member ring, wherein said second 4-7 member ring is optionally substituted by alkyl, alkoxy, nitro, aryl, cyano, amino, halogen, or —NH(C═O)R or —SO₂R where R is alkyl or aryl;
   R₂, R₃ and R₄ are independently hydrogen, alkyl, oxo, amino or together with the organometalic ring to which two of the substituents are attached, a fused 4-7 member ring system wherein said 4-7 member ring is optionally substituted by alkyl, alkoxy, nitro, aryl, cyano, amino, halogen, or —NH(C═O)R or —SO₂R where R is alkyl or aryl; wherein R₄ is not an alkyl; and
   wherein if R₂, R₃ and R₄ are hydrogen and R₁ forms an oxo complex with M, n is 0 then M is Te; or
   if R₂, R₃ and R₄ are hydrogen and R₁ is an oxygen that forms together with the metal an unsubstituted, saturated, 5 member ring, n is 0 then M is Te; or
   if R₁ is an oxo group, and n is 0, R₂ and R₃ form together with the organometalic ring a fused benzene ring, R₄ is hydrogen, then M is Se; or
   if R₄ is an oxo group, and R₂ and R₃ form together with the organometalic ring a fused benzene ring, R₁ is oxygen, n is 0 and forms together with the metal a first 5 member ring, substituted by an oxo group α to R₁, and said ring is fused to a second benzene ring, then M is Te.

In one embodiment, a 4-7 member ring group refers to a saturated cyclic ring. In another embodiment the 4-7 member ring group refers to an unsaturated cyclic ring. In another embodiment the 4-7 member ring group refers to a heterocyclic unsaturated cyclic ring. In another embodiment the 4-7 member ring group refers to a heterocyclic saturated cyclic ring. In one embodiment the 4-7 member ring is unsubstituted. In one embodiment, the ring is substituted by one or more of the following: alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl.

In one embodiment, substituent groups may be attached via single or double bonds, as appropriate, as will be appreciated by one skilled in the art.

According to embodiments herein, the term alkyl as used throughout the specification and claims may include both “unsubstituted alkyls” and/or “substituted alkyls”, the latter of which may refer to alkyl moieties having substituents replacing hydrogen on one or more carbons of the hydrocarbon backbone. In another embodiment, such substituents may include, for example, a halogen, a hydroxyl, an alkoxyl, a silyloxy, a carbonyl, and ester, a phosphoryl, an amine, an amide, an imine, a thiol, a thioether, a thioester, a sulfonyl, an amino, a nitro, or an organometallic moiety. It will be understood by those skilled in the art that the moieties substituted on the hydrocarbon chain may themselves be substituted, if appropriate. For instance, the substituents of a substituted alkyl may include substituted and unsubstituted forms of amines, imines, amides, phosphoryls (including phosphonates and phosphines), sulfonyls (including sulfates and sulfonates), and silyl groups, as well as ethers, thioethers, selenoethers, carbonyls (including ketones, aldehydes, carboxylates, and esters), —CF₃, and —CN. Of course other substituents may be applied. In another embodiment, cycloalkyls may be further substituted with alkyls, alkenyls, alkoxys, thioalkyls, aminoalkyls, carbonyl-substituted alkyls, CF₃, and CN. Of course other substituents may be applied.

In another embodiment, a compound of formula IV is provided, wherein M, R₁ and R₄ are as described above for formula III:

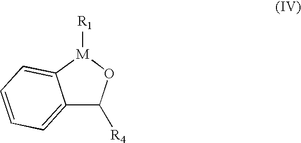

In another embodiment, a compound of formula V is provided, wherein M, R₂, R₃ and R₄ are as described above for formula III:

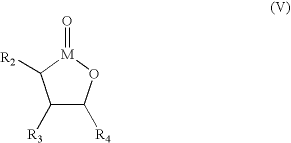

In another embodiment, a compound of formula VI is provided, wherein M, R₂, R₃ and R₄ are as described above for formula III;

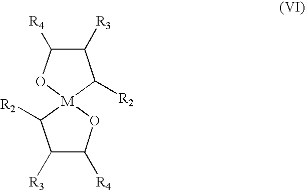

In another embodiment, a compound of formula (VII) is provided, wherein M, R₂ and R₃ are as described above for formula III:

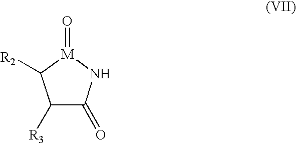

In another embodiment, a compound of formula VIII is provided, wherein M, R₂ and R₃ are as described above for formula III:

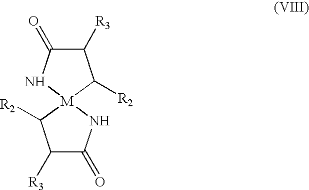

In one embodiment, the compound of formula III, used in the compositions and methods provided herein, is represented by any one of the following compounds or their combinations:

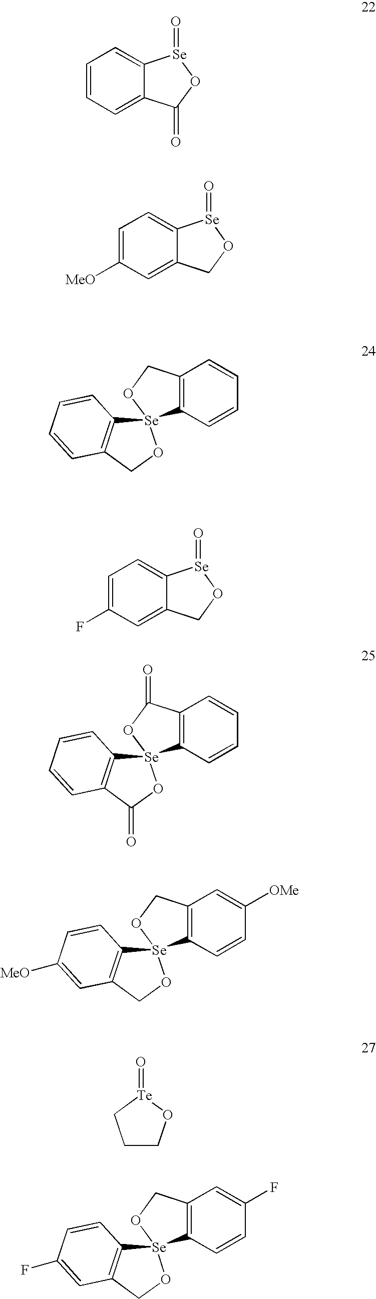

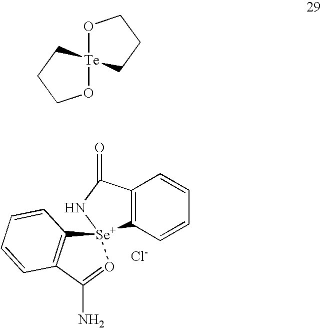

In another embodiment, the antioxidant used in the methods comprising the step of contacting the subject with an effective amount of a composition comprising an antioxidant or its isomer, metabolite, and/or salt therefore is glutathione peroxidase mimetic, is represented by the compound of formula IX:

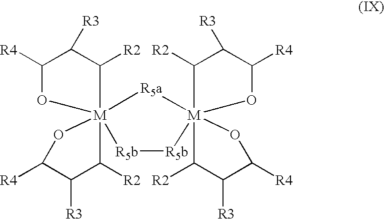

wherein,

### M is Se or Te;

R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 membered ring system, wherein said 4-7 membered ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and

R₅ₐ or R_{5b} is one or more oxygen, carbon, or nitrogen atoms and forms a neutral complex with the chalcogen.

In one embodiment, the compound represented formula (IX), is represented by the compound of formula X:

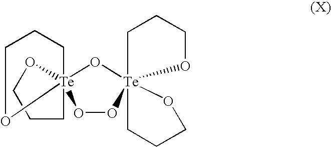

The foregoing compounds are also useful for treating atherosclerosis, and in another embodiment, in subjects with the Hp 2-2 genotype.

In one embodiment, the methods provided herein, using the compositions provided herein, further comprise contacting the subject with one or more additional agent, which is not an antioxidant. In another embodiment, the one or more additional agent, which is not an antioxidant or its isomer, metabolite, and/or salt therefore, nor cholesteryl ester transfer protein inhibitor, is an aldosterone inhibitor. In another embodiment, the additional agent is an angiotensin-converting anzyme. In another embodiment, the additional agent is an angiotensin receptor AT₁ blocker (ARB). In another embodiment, the additional agent is an angiotensin II receptor antagonist. In another embodiment, the additional agent is a calcium channel blocker. In another embodiment, the additional agent is a diuretic. In another embodiment, the additional agent is digitalis. In another embodiment, the additional agent is a beta blocker. In another embodiment, the additional agent is a statin. In another embodiment, the additional agent is a cholestyramine or in another embodiment, the additional agent is a combination thereof.

In one embodiment, the additional therapeutic agent used in the methods and compositions described herein is a statin. In another embodiment, the term “statins” refers to a family of compounds that are inhibitors of 3-hydroxy-3-methylglutaryl coenzyme A (HMG-CoA) reductase, the rate-limiting enzyme in cholesterol biosynthesis. As HMG-CoA reductase inhibitors, in one embodiment, statins reduce plasma cholesterol levels in various mammalian species.

Statins inhibit in one embodiment, cholesterol biosynthesis in humans by competitively inhibiting the 3-hydroxy-3-methyl-glutaryl-coenzyme A (“HMG-CoA”) reductase enzyme. HMG-CoA reductase catalyzes in another embodiment, the conversion of HMG to mevalonate, which is the rate determining step in the biosynthesis of cholesterol. Decreased production of cholesterol causes in one embodiment, an increase in the number of LDL receptors and corresponding reduction in the concentration of LDL particles in the bloodstream. Reduction in the LDL level in the bloodstream reduces the risk of coronary artery disease.

Statins used in the compositions and methods of the invention are lovastatin (referred to as mevinolin in one embodiment, or monacolin-K in another embodiment), compactin (referred to as mevastatin in one embodiment, or ML-236B in another embodiment), pravastatin, atorvastatin (Lipitor) rosuvastatin (Crestor) fluvastatin (Lescol), simvastatin (Zocor), cerivastatin. In one embodiment, the statin used as one or more additional therapeutic agent, is any one of the statins described herein, or in another embodiment, in combination of statins. A person skilled in the art would readily recognize that the choice of statin used, will depend on several factors, such as in certain embodiment, the underlying condition of the subject, other drugs administered, other pathologies and the like.

In one embodiment, the additional agent may be an anti-dyslipidemic agent such as (i) bile acid sequestrants such as, cholestyramine, colesevelem, colestipol, dialkylaminoalkyl derivatives. of a cross-linked dextran; Colestid™; LoCholest™; and Questran™, and the like; (ii) HMG-CoA reductase inhibitors such as atorvastatin, itavastatin, fluvastatin, lovastatin, pravastatin, rivastatin, rosuvastatin, simvastatin, and ZD-4522, and the like; (iii) HMG-CoA synthase inhibitors; (iv) cholesterol absorption inhibitors such as stanol esters, beta-sitosterol, sterol glycosides such as tiqueside; and azetidinones such as ezetimibe, vytorin, and the like; (v) acyl coenzyme A-cholesterol acyl transferase (ACAT) inhibitors such as avasimibe, eflucimibe, KY505, SMP 797, and the like; (vi) CETP inhibitors such as JTT 705, torcetrapib, CP 532,632, BAY63-2149, SC 591, SC 795, and the like; (vii) squalene synthetase inhibitors; (viii) anti-oxidants such as probucol, and the like; (ix) PPAR.alpha. agonists such as beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid™, Lopid™ and Tricor™, and the like; (x) FXR receptor modulators such as GW 4064, SR 103912, and the like; (xi) LXR receptor such as GW 3965, T9013137, and XTCO179628, and the like; (xii) lipoprotein synthesis inhibitors such as niacin; (xiii) renin angiotensin system inhibitors; (xiv) PPAR o partial agonists; (xv) bile acid reabsorption inhibitors, such as BARI1453, SC435, PHA384640, S892.1, AZD7706, and the like; (xvi) PPAR.delta. agonists such as GW 501516, and GW 590735, and the like; (xvii) triglyceride synthesis inhibitors; (xviii) microsomal triglyceride transport (MTTP) inhibitors, such as inplitapide, LAB687, and CP346086, and the like; (xix) transcription modulators; (xx) squalene epoxidase inhibitors; (xxi) low density lipoprotein (LDL) receptor inducers; (xxii) platelet aggregation inhibitors; (xxiii) 5-LO or FLAP inhibitors; and (xiv) niacin receptor agonists.

In one embodiment, the additional agent administered as part of the compositions, used in the methods provided herein, is an anti-platelet agents (or platelet inhibitory agents). The term anti-platelet agents (or platelet inhibitory agents), refers in one embodiment to agents that inhibit platelet function by inhibiting the aggregation, or by adhesion or granular secretion of platelets in other embodiments. In another embodiment, the anti-platelet agents used in the compositions described herein include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. In another embodiment, the anti-platelet agent is IIb/IIIa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., dipyridamole), and pharmaceutically acceptable salts or prodrugs thereof. In another embodiment, the term anti-platelet agents (or platelet inhibitory agents), refers to ADP (adenosine diphosphate) receptor antagonists, which is in one embodiment, an antagonists of the purinergic receptors P₂Y₁ and P₂Y₁₂. In one embodiment, P₂Y₁₂ receptor antagonists is ticlopidine, clopidogrel, or their combination and pharmaceutically acceptable salts or prodrugs thereof.

In another embodiment, the additional agent administered as part of the compositions, used in the methods provided herein, is an anti-hypertensive agents such as (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, bamidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (ix) α/β adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XEN010, and the like; and (xi) -alpha 2 agonists such as lofexidine, tiamenidine, moxonidine, rilmenidine and guanobenz, and the like. Combinations of anti-obesity agents and diuretics or beta blockers may further include vasodilators, which widen blood vessels. Representative vasodilators useful in the compositions and methods of the present invention include, but are not limited to, hydralazine (apresoline), clonidine (catapres), minoxidil (loniten), and nicotinyl alcohol (roniacol).

The renin-angiotensin-aldosterone system (“RAAS”) is involved in one embodiment, in regulating pressure homeostasis and also in the development of hypertension, a condition shown as a major factor in the progression of cardiovascular diseases. Secretion of the enzyme renin from the juxtaglomerular cells in the kidney activates in another embodiment, the renin-angiotensin-aldosterone system (RAAS), acting on a naturally-occurring substrate, angiotensinogen, to release in another embodiment, a decapeptide, Angiotensin I. Angiotensin converting enzyme (“ACE”) cleaves in one embodiment, the secreated decapeptide, producing an octapeptide, Angiotensin II, which is in another embodiment, the primary active species of the RAAS system. Angiotensin II stimulates in one embodiment, aldosterone secretion, promoting sodium and fluid retention, inhibiting renin secretion, increasing sympathetic nervous system activity, stimulating vasopressin secretion, causing a positive cardiac inotropic effect or modulating other hormonal systems in other embodiments.

A representative group of ACE inhibitors consists in another embodiment, of the following compounds: AB-103, ancovenin, benazeprilat, BRL-36378, BW-A575C, CGS-13928C, CL-242817, CV-5975, Equaten, EU-4865, EU-4867, EU-5476, foroxymithine, FPL 66564, FR-900456, Hoe-065, I5B2, indolapril, ketomethylureas, KRI-1177, KRI-1230, L-681176, libenzapril, MCD, MDL-27088, MDL-27467A, moveltipril, MS-41, nicotianamine, pentopril, phenacein, pivopril, rentiapril, RG-5975, RG-6134, RG-6207, RGH-0399, ROO-911, RS-10085-197, RS-2039, RS 5139, RS 86127, RU-44403, S-8308, SA-291, spiraprilat, SQ-26900, SQ-28084, SQ-28370, SQ-23940, SQ-31440, Synecor, utibapril, WF-10129, Wy-44221, Wy-44655, Y-23785, Yissum P-0154, zabicipril, Asahi Brewery AB-47, alatriopril, BMS182657, Asahi Chemical C-111, Asahi Chemical C-112, Dainippon DU-1777, mixanpril, Prentyl, zofenoprilat, 1-(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-6564, idrapril, Marion Merrell Dow MDL-100240, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, saralasin acetate, temocapril, trandolapril, ceranapril, moexipril, quinaprilat and spirapril.

In one embodiment, the terms “aldosterone antagonist” and “aldosterone receptor antagonist” refer to a compound that inhibits the binding of aldosterone to mineralocorticoid receptors, thereby blocking the biological effects of aldosterone. In one embodiment, the term “antagonist” in the context of describing compounds according to the invention refers to a compound that directly or in another embodiment, indirectly inhibits, or in another embodiment suppresses Aldosterone activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the Aldosterone, even when there are spare (unbound) Aldosterone present. In another embodiment, the term “partial antagonist” refers to a compound does not evoke the maximal inhibitory response from the androgen receptor, even when present at concentrations sufficient to saturate the androgen receptors present.

The aldosterone antagonists used in the methods and compositions of the present invention are in one embodiment, spirolactone-type steroidal compounds. In another embodiment, the term “spirolactone-type” refers to a structure comprising a lactone moiety attached to a steroid nucleus, such as, in one embodiment, at the steroid “D” ring, through a spiro bond configuration. A subclass of spirolactone-type aldosterone antagonist compounds consists in another embodiment, of epoxy-steroidal aldosterone antagonist compounds such as eplerenone. In one embodiment, spirolactone-type antagonist compounds consists of non-epoxy-steroidal aldosterone antagonist compounds such as spironolactone. In one embodiment, the invention provides a composition comprising an aldosterone antagonist, its isomer, functional derivative, synthetic analog, pharmaceutically acceptable salt or combination thereof; and a glutathione peroxidase or its isomer, functional derivative, synthetic analog, pharmaceutically acceptable salt or combination thereof, wherein the aldosterone antagonist is epoxymexrenone, or eplerenone, dihydrospirorenone, 2,2;6,6-diethlylene-3oxo-17alpha-pregn-4-ene-21,17-carbolactone, spironolactone, 18-deoxy aldosterone, 1,2-dehydro-18-deoxyaldosterone, RU28318 or a combination thereof in other embodiments.

In one embodiment, Cyclic fluxes of Ca²⁺ between three compartments—cytoplasm, sarcoplasmic reticulum (SR), and sarcomere—account for excitation-contraction coupling. Depolarization triggers in another embodiment, entry of small amounts of Ca²⁺ through the L-type Ca²⁺ channels located on the cell membrane, which in one embodiment, prompts SR Ca²⁺ release by cardiac ryanodine receptors (RyR's), a process termed calcium-induced Ca²⁺ release. A rapid rise in cytosolic levels results in one embodiment, fostering Ca²⁺-troponin-C interactions and triggering sarcomere contraction. In another embodiment, activation of the ATP-dependent calcium pump (SERCA) recycles cytosolic Ca²⁺ into the SR to restore sarcomere relaxation. In another embodiment, Ca²⁺ channel blockers inhibits the triggering of sarcomer contraction and modulate increase in cystolic pressure.

In one embodiment, calcium channel blockers, are amlodipine, aranidipine, bamidipine, benidipine, cilnidipine, clentiazem, diltiazen, efonidipine, fantofarone, felodipine, isradipine, lacidipine, lercanidipine, manidipine, mibefradil, nicardipine, nifedipine, nilvadipine, nisoldipine, nitrendipine, semotiadil, veraparmil, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry, which can be used in the compositions and methods of the invention.

In another embodiment, the β-blocker used in the compositions and methods of the invention is propanalol, terbutalol, labetalol propranolol, acebutolol, atenolol, nadolol, bisoprolol, metoprolol, pindolol, oxprenolol, betaxolol or a combination thereof.

In one embodiment, a diuretic is used in the methods and compositions of the invention. In another embodiment, the diuretic is chlorothiazide, hydrochlorothiazide, methylclothiazide, chlorothalidon, or a combination thereof.

In one embodiment, the additional agent used in the compositions provided herein is a non-steroidal anti-inflammatory drug (NSAID). In another embodiment, the NSAID is sodium cromoglycate, nedocromil sodium, PDE4 inhibitors, leukotriene antagonists, iNOS inhibitors, tryptase and elastase inhibitors, beta-2 integrin antagonists and adenosine 2a agonists. In one embodiment, the NSAID is ibuprofen; flurbiprofen, salicylic acid, aspirin, methyl salicylate, diflunisal, salsalate, olsalazine, sulfasalazine, indomethacin, sulindac, etodolac, tolmetin, ketorolac, diclofenac, naproxen, fenoprofen, ketoprofen, oxaprozin, piroxicam, celecoxib, and rofecoxiband a pharmaceutically acceptable salt thereof. In one embodiment, the NSAID component inhibits the cyclo-oxygenase enzyme, which has two (2) isoforms, referred to as COX-1 and COX-2. Both types of NSAID components, that is both non-selective COX inhibitors and selective COX-2 inhibitors are useful in accordance with the present invention.

In another embodiment, the additional agent administered as part of the compositions, used in the methods provided herein, is a glycation inhibitor, such as pimagedine hydrochloride in one embodiment, or ALT-711, EXO-226, KGR-1380, aminoguanidine, ALT946, pyratoxanthine, N-phenacylthiazolium bromide (ALT766), pyrrolidinedithiocarbamate or their combination in yet another embodiment.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

The term “preventing” refers in another embodiment, to preventing the onset of clinically evident pathologies associated with plaque rupture altogether, or preventing the onset of a preclinically evident stage of pathologies associated with plaque rupture in individuals at risk, which in one embodiment are subjects exhibiting the Hp-2 allele. In another embodiment, the determination of whether the subject carries the Hp-2 allele, or in one embodiment, which Hp allele, precedes the methods and the step of administration of the compositions of the invention.

In another embodiment, the route of administration in the step of contacting in the methods of the invention, using the compositions described herein, is optimized for particular treatments regimens. If chronic treatment of plaques is required, in one embodiment, administration will be via continuous subcutaneous infusion, using in another embodiment, an external infusion pump. In another embodiment, if acute treatment of plaque rupture is required, such as in one embodiment, in the case of intraplaque hemorrhage, then intravenous infusion is used.

In one embodiment, the compositions provided herein are administered in conjunction with other therapeutical agents. Representative agents that can be used in combination with the compositions of the invention are agents used to treat diabetes such as insulin and insulin analogs (e.g. LysPro insulin); GLP-1 (7-37) (insulinotropin) and GLP-1 (7-36)-NH.sub.2; biguanides: metformin, phenformin, buformin; .alpha.2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glypizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; .alpha.-glucosidase inhibitors: acarbose, miglitol, emiglitate, voglibose, MDL-25,637, camiglibose, MDL-73,945;, .beta.-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan®)) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (Symlin™), AC2993, glycogen phosphorylase inhibitor and nateglinide. Any combination of agents can be administered as described hereinabove.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### Construction of a Murine Hp 2 Allele

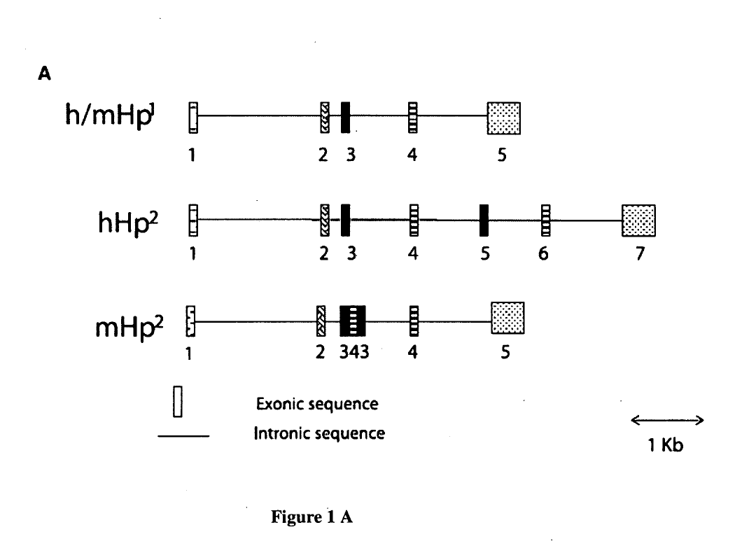

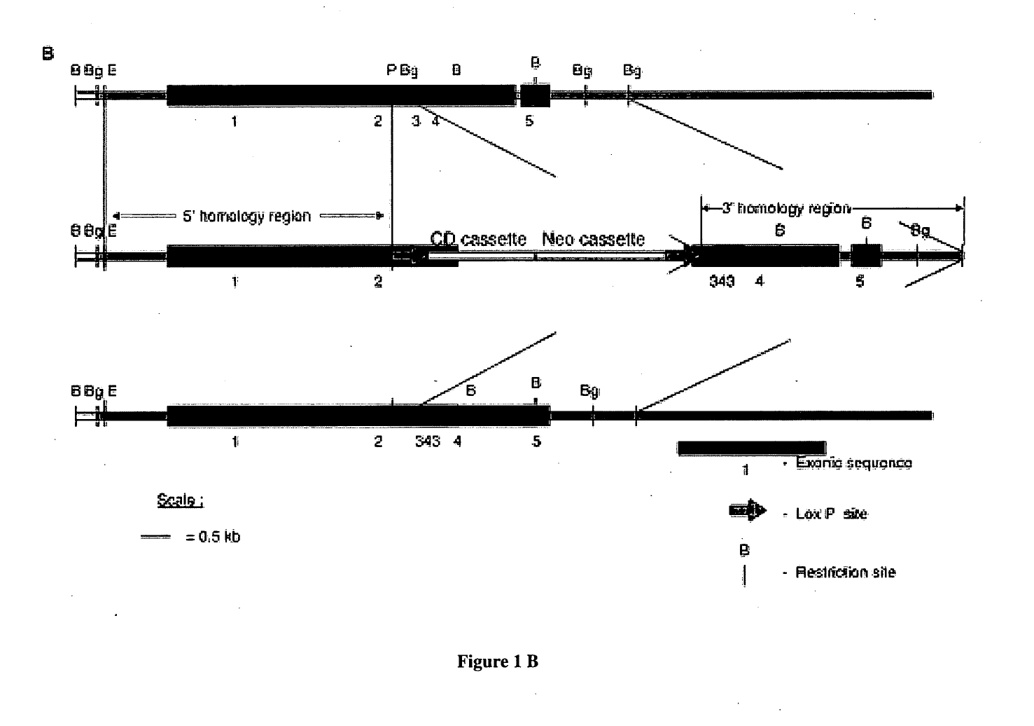

The rationale and cloning strategy for producing a murine Hp 2 allele and targeting its insertion by homologous recombination are provided in an online supplement. The genomic organization of the human Hp locus is shown in FIG. 1A. FIG. 1B provides a map of the murine Hp locus before and after gene targeting.

### Care of Mice and Harvesting of Tissues

These studies were approved by the Animal Care Committee of the Technion. Mice were fed a normal diet and euthanized at 9 months.

Total serum cholesterol (Roche), triglycerides (Roche), and highdensity lipoprotein (Biosystems, Barcelona) were measured enzymatically. Serum Hp was measured based on the acid stable peroxidase activity of the Hp-Hb complex (Tridelta, Bray, UK).

The aortic arch was fixed in 4% formaldehyde, embedded in paraffin, and sectioned using a Leica RM 2155 microtome. Total plaque area, lipid area, and minimum cap thickness were quantified as previously described. [Moreno P R, Purushothaman K R, Fuster V, O'Connor W N. Intimomedial interface damage and adventitial inflammation is increased beneath disrupted atherosclerosis in the aorta: implications for plaque vulnerability. Circulation. 2002; 105:2504-2511, and Moreno P R, Lodder R A, Purushothaman K R, Charash W E, O'Connor W N, Muller J E. Detection of lipid pool, thin fibrous cap, and inflammatory cells in human aortic atherosclerotic plaques by near-infrared spectroscopy. Circulation. 2002; 105:923-927].

### Iron Deposition

Iron deposition in the plaque was identified using Perl's stain and quantified by measuring the percentage of plaque area staining black.

### Lipid Peroxidation

Lipid peroxidation was evaluated using 4-hydroxynonenal (4-HNE) and ceroid.

### Macrophage Accumulation

Immunohistochemical localization of macrophages was performed using standard methods.

### Statistical Analysis

All results, with the exception of total plaque and lipid core area, are reported as the mean±SEM with differences between groups determined by a 2-tailed t test. Data for total plaque and lipid core area are reported as the 25th/50th/75th percentile with differences between groups determined by the Mann-Whitney test. A value of P≦0.05 was considered significant.

### Example 1

### Generation of a Murine Hp 2 Allele

The murine Hp 2 allele was engineered to have an intragenic duplication of exons 3 and 4, analogous to that found in the human Hp 2 allele (FIGS. 1A and 1B). Once generated, the murine Hp 2 allele was used to replace the normal mouse Hp 1 allele by homologous recombination.

### Example 2

### The Shape and Size of the Murine Hp 2 Allele Protein Product is Similar to the Human Hp 2 Allele Protein Product

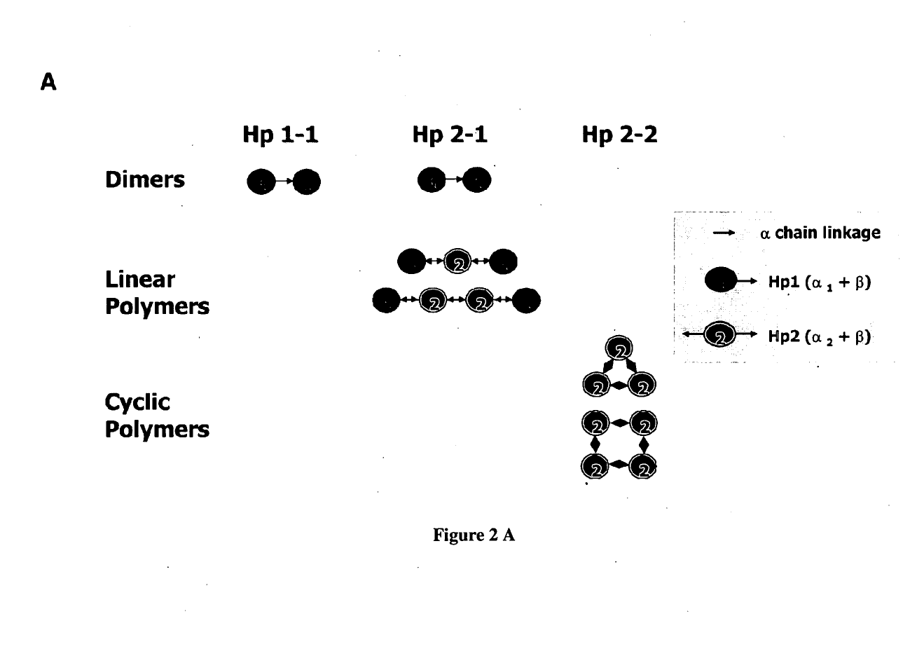

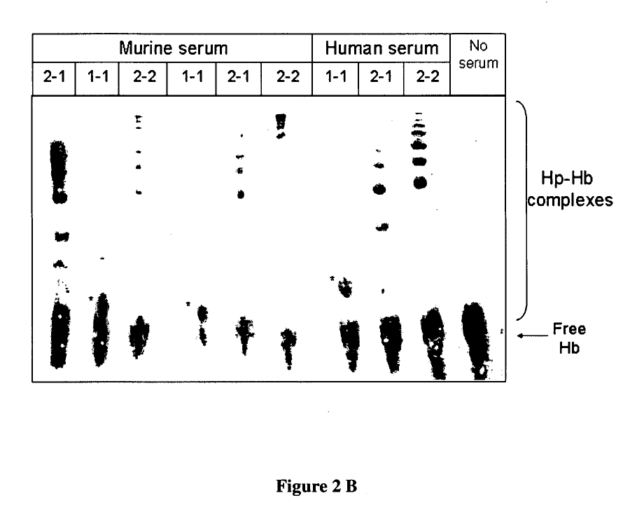

FIG. 2A shows schematically the difference as visualized by electron microscopy between the shape and size of Hp polymers found in humans with the Hp 1-1, 2-1, or 2-2 genotypes. Hp is synthesized as a single polypeptide that is proteolytically cleaved to give an α-chain (9 or 16 Kd derived from exons 1 to 4 or 1 to 6 for the 1 or 2 allele, respectively) and a beta chain (45 Kd derived from exon 5 or exon 7 for the 1 or 2 allele, respectively). The Hp α-beta monomer is covalently linked via disulfide bonds with other Hp monomers in an Hp genotype-dependent fashion. This is because the cysteine residues responsible for Hp polymerization are present in the region of the Hp gene duplicated in the Hp 2 allele. An Hp monomer derived from the Hp 1 allele can be cross-linked with only one Hp monomer (it is monovalent) to form an Hp dimer. However, the Hp monomer derived from the Hp 2 allele is cross-linked with 2 Hp monomers (it is bivalent). In individuals with only the Hp 2 protein, the plasma Hp molecules are all cyclic polymers. In heterozygotes, Hp polymers are dimers, trimers, and quatermers that are linear. These different polymeric structures can be easily visualized by taking advantage of the interaction of Hp with Hb and the peroxidase activity of Hb and Hb-Hp complexes. Electrophoresis on a nondenaturing polyacrylamide gel of Hb-enriched serum followed by immersion of the gel in 3,3′,5,5′-tetramethylbenzidine (forming a precipitate in the gel at the site of peroxidase activity) produces a signature banding pattern characteristic for each Hp genotype. In such gels, a single rapidly migrating band is seen in serum derived from Hp 1-1 individuals, corresponding to the Hp dimer, whereas more slowly migrating bands are seen in Hp 2-1 or Hp 2-2 individuals corresponding to the higher order linear and cyclic polymers present in these individuals (FIG. 2B). The cysteine residues of murine and human Hp are 100% conserved, and therefore the gene duplication event, which we have introduced in the murine Hp allele, would be predicted to result in a similar polymerization profile as the human Hp 2 allele. As demonstrated in FIG. 2B, the banding pattern in a nondenaturing polyacrylamide gel of Hb-enriched serum from mice with the Hp 2 allele is remarkably similar to humans with the Hp 2 allele demonstrating that the gene duplication we have produced in the murine Hp 2 allele produces higher-order Hp polymers similar to those seen in humans with the Hp 2 allele (FIG. 2B). Furthermore, the serum concentration of Hp protein was similar in mice with Hp 1-1 and Hp 2-2 genotypes (0.92±0.45 versus 1.10±0.37, P=0.66) and was similar to the Hp concentration reported for human serum.

### Example 3

### Morphometric Measurements of the Atherosclerotic Plaques

18 plaques from 9 C57B16/6J ApoE^{-/-} Hp1-1 mice and 15 plaques from 6 C57B16/6J ApoE^{-/-} Hp2-2 mice were characterized and compared. There was no significant difference between the Hp 1-1 and Hp 2-2 mice with regard to age, weight, total serum cholesterol (432±67 mg/dL versus 353±45 mg/dL, P=0.34), triglycerides (143±20 mg/dL versus 101±12 mg/dL, P=0.15), or high-density lipoprotein cholesterol (22.3±4.6 mg/dL versus 21.5±4.4 mg/dL, P=0.83). Fibrous cap thickness, plaque area, and lipid core area in Hp 1-1 and Hp 2-2 mice are presented in Table I. There was no significant difference in plaque or lipid core area between Hp 1-1 and Hp 2-2 mice. There was a qualitative trend showing decreased cap thickness in plaques from Hp 2-2 mice.

TABLE I
Morphometric Properties of Plaques in Hp 1-1 and Hp 2-2 Mice

| | | Cap Thickness | Plaque Area | Lipid Core |
|---|---|---|---|---|
| Genotype | n | (um) | (um²) | (um²) |
| apoE^{-/-} | 18 | 19.1 ± 2.2 | 0.018/0.033/0.144 | 0.006/0.017/0.035 |
| Hp 1-1 | | | | |
| apoE^{-/-} | 15 | 15.0 ÷ 1.7 | 0.027/0.051/0.084 | 0,008/0.022/0.035 |
| Hp 2-2 | | | | |

where: n indicates total number of plaques analyzed. For cap thickness, the mean±SEM is shown. For plaque area and lipid core area the quartile values (25th/50th/75th percentiles) are shown. There was no significant difference in cap thickness (P=0.25), plaque area (P=0.76), or lipid core area (P=0.73) between Hp 1-1 and Hp 2-2 mice.

### Example 4

### Generation of a Murine Hp 2 Allele

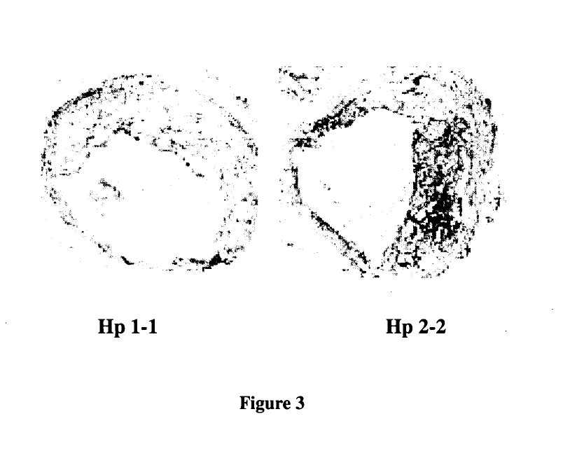

Previous in vitro studies have suggested that hemoglobin released from microvascular hemorrhages within the plaque would be cleared more slowly in Hp 2-2 as compared with Hp 1-1 plaques. Consistent with this hypothesis, significantly increased iron staining, calculated as the percentage of the total plaque area, was found in Hp 2-2 plaques as compared with Hp 1-1 plaques (2.18±0.26% versus 0.94±0.25%, n=10, P=0.008) (FIG. 3).

### Example 5

### Increased Lipid Peroxidation in Hp 2-2 Plaques

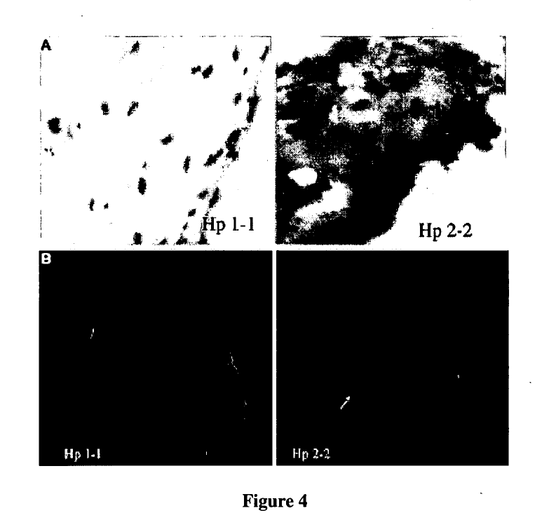

Plaques were assessed for 4-HNE, a major end-product of lipid peroxidation, and ceroid, a mixture of autofluorescent oxidized lipid and protein. Markedly greater 4-HNE (FIG. 4A) and ceroid (autofluorescence) (FIG. 4B) were found in the plaques of Hp 2-2 as compared with Hp 1-1 mice.

### Example 6

### Increased Macrophage Accumulation in Hp 2-2 Plaques

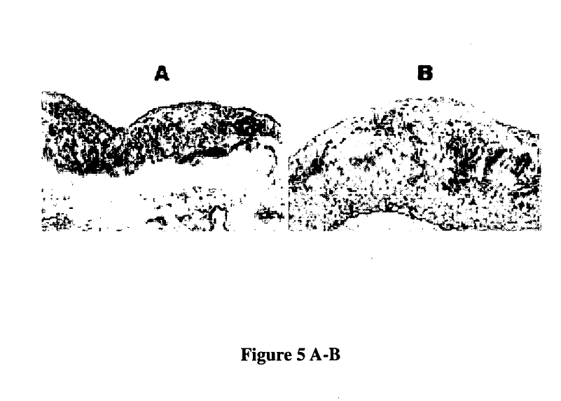

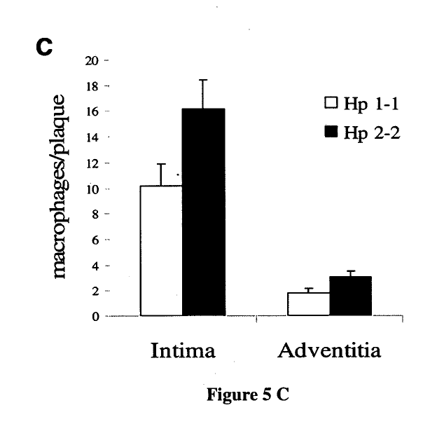

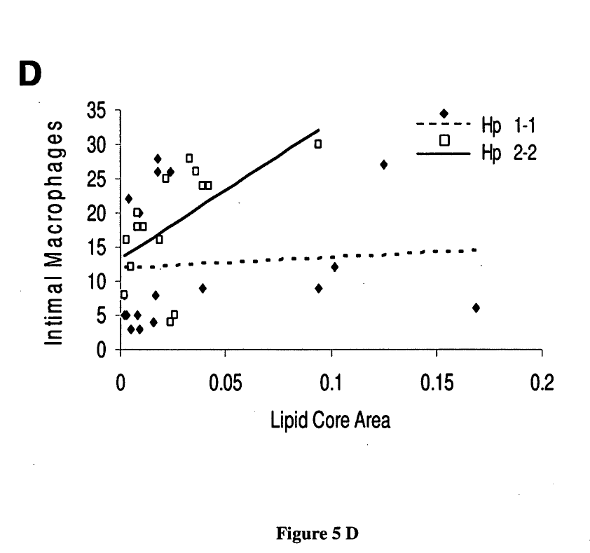

The intima and adventitia of atherosclerotic plaques from Hp 2-2 mice were were found to contain significantly more macrophages as compared with plaques from Hp 1-1 mice (FIG. 5).

### Example 7

### Correlation Between Lipid Core Size and Inflammation in Hp 2-2 Plaques but not in Hp 1-1 Plaques

Oxidized lipid within the core of the plaque may act as an inflammatory stimulus. The finding described in the examples above that although there was no significant difference in the lipid core area between Hp 1-1 and Hp 2-2 mice, macrophage accumulation in the Hp 2-2 plaques was significantly greater was intriguing. Therefore the correlation between the lipid area and macrophage accumulation was examined. A significant correlation between the size of the lipid core and the number of intimal macrophages in plaques from Hp 2-2 mice (correlation coefficient r=0.57, P=0.01) was found, whereas no correlation was found between the size of the lipid core and the number of macrophages in plaques from Hp 1-1 mice (correlation coefficient r=0.08, P=0.38) (FIG. 5D).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of determining susceptibility of a subject to a plaque rupture comprising the step of obtaining a biological sample from the subject; and determining the subject's haptoglobin allelic genotype, whereby a subject expressing the Hp-2-2 genotype is susceptible to plaque rupture.

2. The method of claim 1, whereby said step of determining said haptoglobin genotype is effected by a method selected from a signal amplification method, a direct detection method, detection of at least one sequence change, immunological method or a combination thereof.

3. The method of claim 2, whereby said signal amplification method amplifies a molecule selected from the group consisting of a DNA molecule and an RNA molecule.

4. The method of claim 2, whereby said signal amplification method is selected from the group consisting of PCR, LCR (LAR), Self-Sustained Synthetic Reaction (3SR/NASBA) and Q-Beta (Qβ) Replicase reaction.

5. The method of claim 2, whereby said direct detection method is selected from the group consisting of a cycling probe reaction (CPR) and a branched DNA analysis.

6. The method of claim 2, whereby said detection of at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

7. The method of claim 2, whereby step of determining said haptoglobin genotype is effected by an immunological detection method.

8. The method of claim 7, whereby said immunological detection method is a radio-immunoassay (RIA), an enzyme linked immunosorbent assay (ELISA), a western blot, an immunohistochemical analysis, or fluorescence activated cell sorting (FACS).

9. The method of claim 1, whereby the subject is diabetic.

10. The method of claim 1, whereby the plaque rupture results in intraplaque hemorrhage

11. A method of treating, inhibiting or suppressing, or reducing symptoms associated with a plaque rupture in a subject, comprising the step of contacting the subject with an effective amount of a composition comprising an antioxidant or its isomer, metabolite, and/or salt therefore, thereby treating plaque rupture, inhibiting or suppressing a plaque rupture, or reducing symptoms associated with plaque rupture.

12. The method of claim 11, whereby said subject is diabetic.

13. The method of claim 11, whereby said antioxidant or its isomer, metabolite, and/or salt therefore, is a glutathione peroxidase mimetic represented by the compound of formula I:

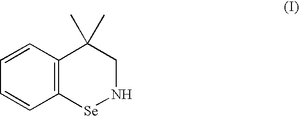

14. The method of claim 11, whereby said antioxidant or its isomer, metabolite, and/or salt therefore, is a benzisoselen-azoline or -azine derivatives of glutathione peroxidase mimetic, represented by the following general formula II: 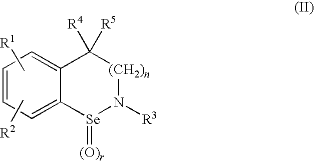
wherein R¹=R²=hydrogen; lower alkyl; OR⁶; —(CH₂)ₘ NR⁶R⁷; —(CH₂)_{q}NH₂; —(CH₂)ₘ NHSO₂ (CH₂)₂ NH₂; —NO₂; —CN; —SO₃ H; —N⁺ (R⁵)₂ O⁻; F; Cl; Br; I; —(CH₂)ₘ R⁸; —(CH₂)ₘ COR⁸; —S(O)NR⁶ R⁷; —SO₂ NR⁶ R⁷; —CO(CH₂)ₚ COR⁸; R⁹; R³=hydrogen; lower alkyl; aralkyl; substituted aralkyl; —(CH₂)ₘ COR⁸; —(CH₂)_{q}R⁸; —CO(CH₂)ₚ COR⁸; —(CH₂)ₘ SO₂ R⁸; —(CH₂)ₘ S(O)R⁸;
R⁴=lower alkyl; aralkyl; substituted aralkyl; —(CH₂)ₚ COR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae: 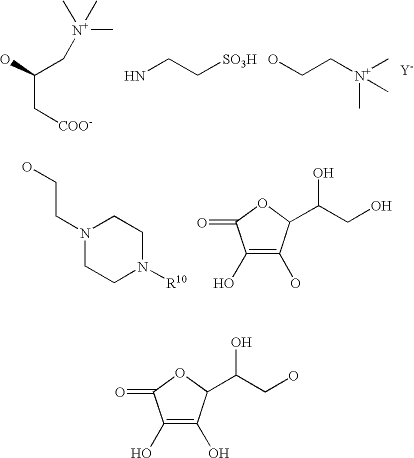
R¹⁰=hydrogen; lower alkyl; aralkyl or substituted aralkyl; aryl or substituted aryl; Y⁻ represents the anion of a pharmaceutically acceptable acid;
n=0, 1; m=0, 1, 2; p=1, 2, 3; q=2, 3, 4; and
r=0, 1.

15. The method of claim 11, whereby the antioxidant or its isomer, metabolite, and/or salt therefore is represented by the compound of formula III: 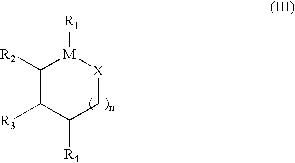
wherein,
the compound of formula 1 is a ring; and
X is O or NH
M is Se or Te
n is 0-2
R¹ is oxygen; and forms an oxo complex with M; or
R¹ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 member ring, substituted by an oxo group α to R₁, and said ring is fused to a second benzene ring, then M is Te.

16. The method of claim 15, whereby the compound of formula III is represented by the compound of formula IV: 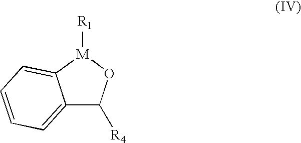
wherein, M, R₁ and R₄ are as described above.

17. The method of claim 15, whereby the compound of formula III is represented by the compound of formula V: 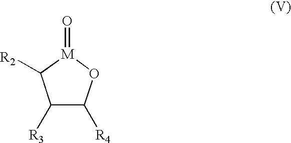
wherein, M, R₂, R₃ and R₄ are as described above.

18. The method of claim 15, whereby the compound of formula III is represented by the compound of formula VI: 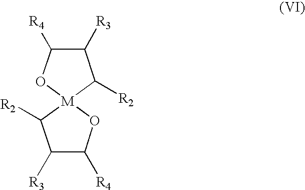
wherein, M, R₂, R₃ and R₄ are as described above.

19. The method of claim 15, whereby the compound of formula III is represented by the compound of formula VII: 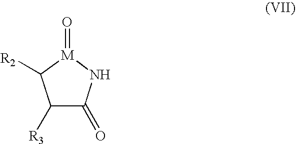
wherein, M, R₂, and R₃ are as described above.

20. The method of claim 15, whereby the compound of formula III is represented by the compound of formula VIII: 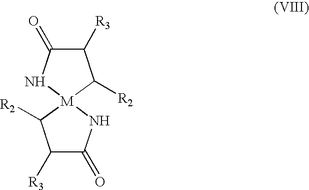
wherein, M, R₂, and R₃ are as described above.

21. The method of claim 15, whereby the compound of formula III is represented by the compounds:

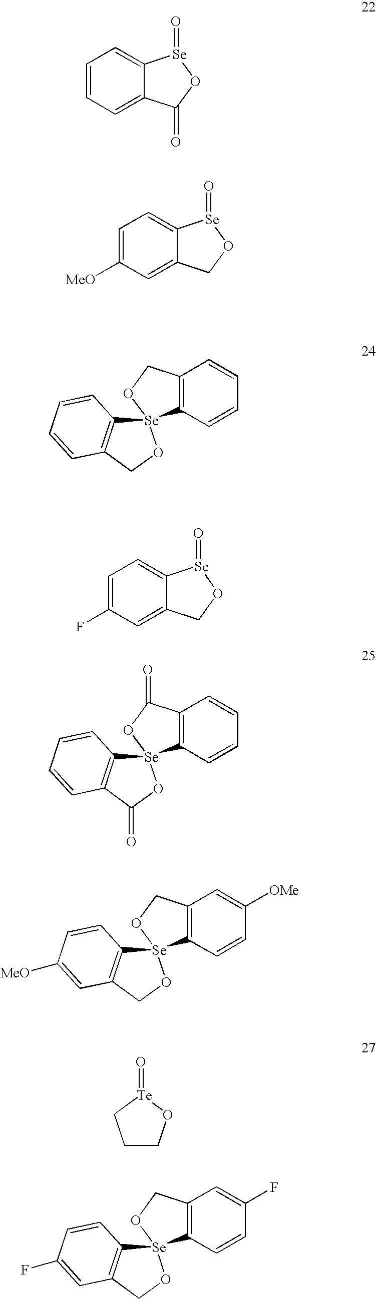

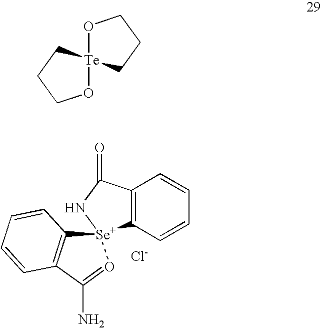

22. The method of claim 11, whereby the antioxidant or its isomer, metabolite, and/or salt therefore, is represented by the compound of formula IX: 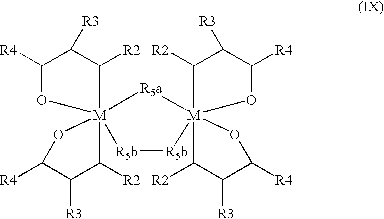
wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

23. The method of claim 22 whereby the compound of formula IX is represented by the compound of formula X:

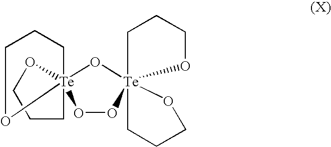

24. The method of claim 11, whereby the step of contacting is via oral, intravenous, intraarterial, intramuscular, subcutaneous, parenteral, transmucosal, transdermal, intracranial, or topical administration.

25. The method of claim 11, comprising contacting the subject with one or more additional agent, which is not an antioxidant or its isomer, metabolite, and/or salt therefore.

26. The method of claim 25, whereby the one or more additional agent not an antioxidant or its isomer, metabolite, and/or salt therefore, is an aldosterone inhibitor, and angiotensin-converting anzyme, an angiotensin receptor AT₁ blocker (ARB), an angiotensin II receptor antagonist, a calcium channel blocker, a diuretic, digitalis, a beta blocker, a statin, a cholestyramine, a NSAID, a glycation inhibitor or a combination thereof.

27. The method of claim 11, whereby the antioxidant or its isomer, metabolite, or salt therefore is vitamin E, butylated hydroxytoluene (BHT), butylated hydroxyanisole (BHA), propyl gallate (PG), dodecylgallate, tert-butylhydroquinone (TBHQ), dihydrolipoic acid, prostaglandin B₁ oligomers, 2-aminomethyl-4-tert-butyl-6-iodophenol, 2-aminomethyl-4-tert-butyl-6-propionylphenol, 2,6-di-tert-butyl-4-[2′-thenoyl]phenol, N,N′-diphenyl-p-phenylenediamine, ethoxyquin, probucol, 5-[[3,5-bis(1,1-dimethylethyl)-4-hydroxyphen-yl]methylene]-3-(dimethylamino)-4-thiazolidinone, 5-[[3,5-bis(1,1-dimethylethyl)-4-hydroxyphenyl]meth-ylene]-3-(methylamino)-4-thiazolidinone, D-myoinositol-1.2.6-trisphosphate, nordihydroguaiaretic acid, deferoxamine mesylate, tirilazad mesylate, trimetazidine, N,N′-dimethylthiourea, 2-(2-hydroxy-4-methylphenyl)aminothiazolehydrochloride, thioctic acid or 2-L-oxothiazolidine.

28. The method of claim 11 wherein the subject has the haptoglobin 2-2 genotype.

29. A method of treating, inhibiting or suppressing, or reducing symptoms associated with an intraplaque hemorrhage in a subject, comprising the step of contacting the subject with an effective amount of a composition comprising an antioxidant or its isomer, metabolite, and/or salt therefore, thereby treating intraplaque hemorrhage, inhibiting or suppressing intraplaque hemorrhage, or reducing symptoms associated with intraplaque hemorrhage.
